(19) United States
(12) Patent Application Publication
(10) Pub. No.: US 2007/0031475 A1
(43) Pub. Date: Feb. 8, 2007

(54) Nutritional composition with unsaturated fatty acids and trace elements

(75) Inventors: Martinas Kuslys, Grosshoechstetten (CH); Andre Francois Charles Sohier, St Legier (CH)

Correspondence Address: BELL, BOYD & LLOYD LLC, P. O. BOX 1135, CHICAGO, IL, 60690-1135, US

(73) Assignee: Nestec S.A., Vevey (CH)

(21) Appl. No.: 10/572,420

(22) PCT Filed: Sep. 23, 2004
(86) PCT No.: PCT/EP04/10672

§ 371 Date: Jun. 5, 2006

(30) Foreign Application Priority Data
Sep. 26, 2003 (EP) 03021847.3

(51) Int. Cl. A61K 47/00; A61K 31/22; A61K 31/202; A61K 33/26

(52) U.S. Cl. 424/439; 424/646; 514/547; 514/560

(57) ABSTRACT

The present invention relates to a powdered nutritional composition, which comprises at least long-chain polyunsaturated fatty acids and trace elements, such as iron, besides optional further constituents, such as macro- and micronutrients. Due to encapsulation of the iron source with or in a fat, wax or mixtures thereof, lipid-oxidation may be largely reduced and fishy off-flavour formation is prevented. Thus, a powdered composition with extended shelf-life may be obtained.

The present invention relates to a powdered nutritional composition comprising unsaturated fatty acids, an antioxidant and at least one encapsulated trace element, to a method for preparing the composition and to a method of reducing off-flavour development in a powdered nutritional composition.

### THE BACKGROUND ART

The addition of oils rich in LC-PUFAs and/or MUFAs (long chain poly- and/or monounsaturated fatty acids) to nutritional compositions has been recognised as beneficial for some time. For example, it is generally held mandatory to add LC-PUFAs to nutritional compositions for pre-term infants. As a matter of fact, LC-PUFAs are found in mother's milk.

In addition, LC-PUFAs and MUFAs are also added to other than infant nutritional compositions, mainly to optimise the lipid profile for nutritional reasons.

The double bonds present in unsaturated fatty acids, however, make them prone to oxidation, which is further increased by light (photo-oxidation), and by a self-catalytic reaction triggered by free radicals present in the environment (autooxidation).

Due to the oxydized and typically “fishy” and extremely dominant off-flavours that go hand in hand with the oxidation of certain unsaturated fatty acids, such as LC-PUFAs, it is a general problem to provide LC-PUFAs and MUFAs within a food product, while at the same time reducing or avoiding any negative impact on flavour or taste.

In particular, it was found that certain nutrients, especially trace elements, may catalyse or increase the oxidation and its undesired side effects. It is, therefore, an objective to provide a nutritional composition, which comprises trace elements, such as iron, and unsaturated fatty acids, whereby the oxidation of the unsaturated fatty acids is reduced to the largest possible extent.

It is an objective to provide a shelf-stable, powdered nutritional composition comprising an iron and/or copper source and unsaturated fatty acids, in particular LC-PUFAs, which does not develop a bad or fishy taste after continued shelf life or after reconstitution.

### SUMMARY OF THE INVENTION

Surprisingly, the inventor of the present invention found that by mixing a powder component which comprises at least one trace element encapsulated with or in a fat, wax or mixtures thereof with a further powder component which comprises unsaturated fatty acids and an antioxidant, side-effects linked to fat oxidation may be strongly reduced, even though both components are homogenously intermingled to form a single powdered nutritional composition. Remarkably, products with significantly longer shelf-life and better taste could be obtained.

Accordingly, in a first aspect, the present invention provides a powdered nutritional composition, which contains a powder component (a) comprising unsaturated fatty acids and an antioxidant, and, a further powder component (b) comprising at least one trace element encapsulated with or in a fat, wax or mixtures thereof.

In a further aspect, the present invention provides a method for preparing a powdered nutritional composition comprising unsaturated fatty acids and trace-elements, comprising the steps of:
preparing a hydrated mixture comprising unsaturated fatty acids and an antioxidant to obtain a component (a),
drying the hydrated mixture to obtain a powder component (a),
adding a powder component (b), which comprises at least one trace element encapsulated with or in a fat, wax or mixtures thereof.

In another aspect, the present invention provides a method of reducing off-flavour development in a powdered nutritional composition which contains LC-PUFAs and an iron source by protecting the LC-PUFA in a powder component (a) comprising an antioxidant and maintaining the iron source encapsulated with or in a fat, wax or mixtures thereof outside the individual particles of the powder component.

In the figures,

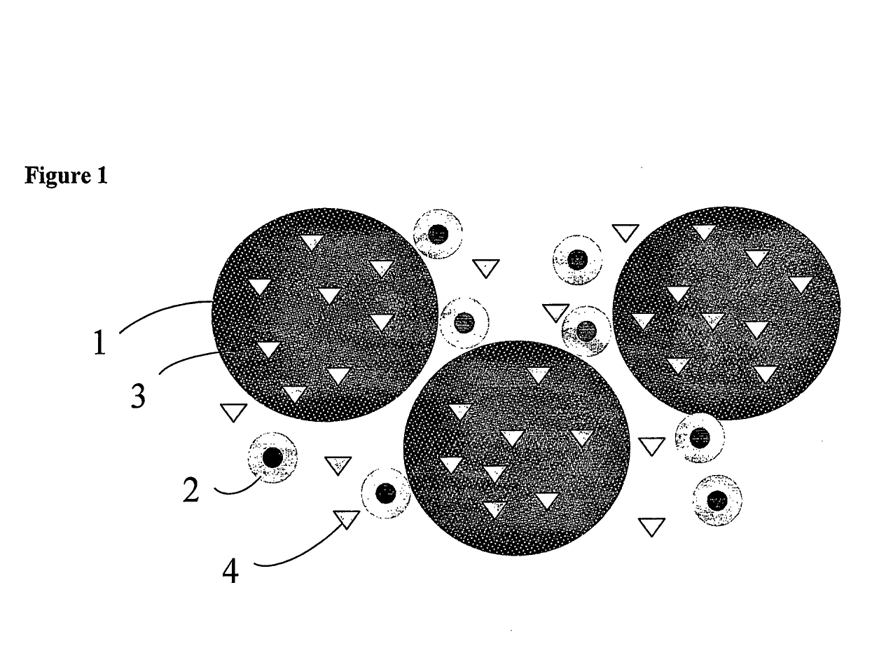

FIG. 1 schematically shows the particles of the powdered composition according to the present invention.

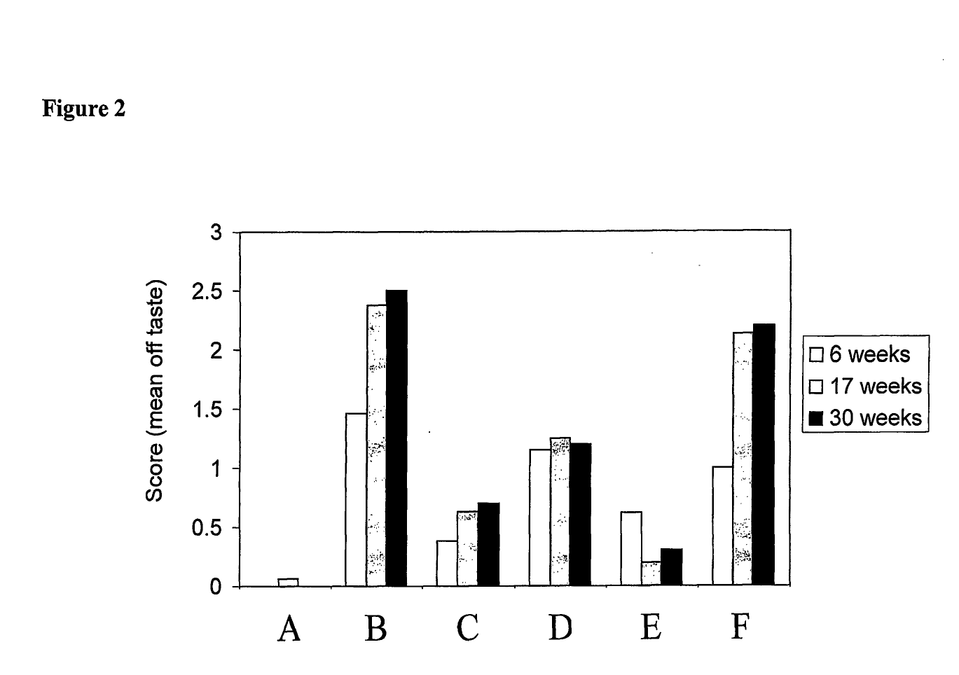

FIG. 2 shows off-taste developed in different nutritional compositions comprising LC-PUFAs and FeSO₄.

### DETAILED DESCRIPTION OF THE INVENTION

Within the context of this specification the word “comprises” or “contains” is taken to mean “includes, among other things”. It is not intended to be construed as “consists of only”.

Within the context of the present invention, the term “powder component” is intended to include a powder that has powder-particles formed entirely by one component, and other particles, formed by the other component. In this understanding, the present invention comprises at least two different kind of particles, corresponding to powder-component (a) and (b), respectively.

However, in an alternative embodiment, the term powder-component also includes a powder comprising individual powder-particles including themselves several powder components. For example, the powder-components (a) and (b) may be present in the form of physically separated parts within a single powder-particle. In this understanding, the term powder-component could be equated to “particle-component”.

In the context of the present invention, the term “dry-mixed” indicates that powdered trace elements or vitamin C, or any other ingredient, was added to the already (spray-)dried, powdered composition at the end in a dried form. “Wet-mixed” means that the specific ingredient of the nutritional composition was hydrated together with other ingredients before homogenisation, heat treatment and drying.

The nutritional composition according to the present invention may be any powdered product, which contain unsaturated fatty acids. Hence, it may be any nutritional composition, designed for any consumer or any patient population.

For example, it may be a pre-term infant formula, a starter infant formula, a follow-up infant formula, a pediatric formula, a formula for pregnant or lactating women, a formula for elderly people or for any patients in need of a specific nutritional needs, during hospitalisation, for example. These examples merely reflect the general applicability of the inventive concept according to the present invention.

The term “hydrated”, in the context of the present invention, means mixed with water.

In the context of the present invention, all percentages are percent by weight, unless otherwise indicated. If percentages are by weight of the nutritional composition, they refer to the weight of the powdered, dehydrated composition.

The nutritional composition according to the invention comprises at least one trace element. In a preferred embodiment, the trace element is iron, cobalt, cupper or mixtures thereof, in a bio-available form. Preferably, the trace elements are iron and/or copper.

Preferably, the trace element is present in a bio-available and, of course, in a nutritionally safe form. Preferably, the trace elements are in a hydrophobic (uncharged) form. For example, if the trace element is Fe, it may be present in the form of FeSO₄, Ferrous lactate, Ferrous Ammonium Phosphate. The skilled person can easily chose a commercially available source of iron suitable as a trace element in a nutritional composition.

According to the present invention, the at least one trace element is encapsulated with or in a fat, wax or mixtures thereof, which constitutes component (b) according to the present invention. Component (b) comprises particles having an encapsulation matrix as well as the encapsulated trace elements.

Preferably, the lipid for encapsulating the trace element is oxidation stable, meaning that is essentially free of unsaturated fatty acids or comprises predominantly saturated fatty acids.

For example, the matrix comprises a fat or wax and/or other lipids that has a high melting point. For example, the melting point is above 40° C. Preferably, the melting point is above 50°, more preferably above 60° C. and most preferably above 65° C. High melting points are preferred because they prevent melting of the fat upon reconstitution of the powdered composition according to the invention with hot water or milk, for example.

On the other hand, oxidation stable lipids, such as fat or wax preferably has physical properties that allow for liberation of the trace element during gastro-intestinal passage.

The encapsulated trace elements may be commercially obtained already in an encapsulated form. Preferred products and their suppliers are named below:

Product name: Vitashure 484® (16% Fe as FeSO₄)

Balchem Corporation

P.O. Box 175

State Hill, N.Y. 10973, USA

Product Name: Encapsulated iron (16% Fe as FeSO₄)

Dr. Paul Lohmann

Hauptstrasse 2

D-31860 Emmerthal

Germany

Product name: P-3622 (7.3% Fe as Ferrous lactate)

Bio Dar Ltd.

Yavne Technology Park

P.O. Box 344

Yavne 81103, Israel

Typical particle sizes for the products obtainable from Dr. P. Lohman indicated above are in the range of 150 μm to 350 μm.

Preferably, the particles of encapsulated trace elements, which constitute component (b) according to the invention, comprise 30-70% of an oxidation stable lipid and 70-30% of a salt comprising the trace element or several trace elements. The particles may further comprise up to 10% of an antioxidant. More preferably, the particles comprise 40-60% of the oxidation stable lipid and 60-40% of the salt or salts.

Preferably, the particles constituting powder-component (b) of the invention comprise ferrous iron, that is FeII-ions, in amounts of about 6-30%, preferably 7-25%, more preferably 10-20%, most preferably 13-18% in percent by weight of component (b).

Component (b) comprising at least one encapsulated trace element may be added to the nutritional composition, in percent by weight of the dehydrated nutritional composition, in an amount of 0.01-1%, preferably 0.015-0.5%, more preferably 0.02-0.3%, most preferably 0.02-0.15%.

Preferably, the powdered composition according to the invention comprises 1-50 mg, more preferably 2-30 mg, most preferably 3-24 mg of iron per 100 g of the powdered composition.

If the encapsulated trace element is copper, the same of above applies, with the difference that the added amounts are 2-5 times lower.

Component (a) of the composition according to the present invention comprises unsaturated fatty acids. For example, component (a) comprises oils or fats rich in unsaturated fatty acids such as LC-MUFAs or PUFAs. Preferably, component (a) of the composition comprises oils rich in LC-PUFAs. For example, the component (a) comprises omega-3 (ω-3) and omega 6 (ω-6) fatty acids.

Preferably, the oil rich in unsaturated fatty acids comprises 1-40%, more preferably 5-40%, most preferably 5-15% by weight of LC-PUFAs.

Preferably, component (a) comprises fatty acids selected from the group of docosahexaenoic acid (DHA, C22:6), arachidonic acid (ARA, C20:4), linoleic acid (LA, C18:2), α-linolenic acid, dihomogammalinolenic acid (DHGLA), eicosapentaenoic acid (EPA) and mixtures of these.

Preferably, the unsaturated fatty acid is DHA, more preferably it is a mixture comprising DHA and ARA; DHA and EPA; or DHA, ARA, and EPA.

The skilled person generally knows sources of unsaturated fatty acids. Typical sources of DHA, for example, are fish oil or oils from micro organisms, such as Cryptecodinum cohnii. EP 0 515 460, for example, discloses a method for obtaining oil rich in DHA which is present in the biomass of cultivated dinoflagellates. WO 02/072742 discloses oils rich in DHA, ARA, DHGLA and EPA.

A typical source of ARA, for example, is egg lecithin or biomass of fermentation processes (Mortierella alpina), the latter may be obtained according to the process disclosed in EP 0 568 608.

In terms of the entire powdered composition comprising components (a) and (b), the composition may comprise, in percent by weight, 0.01-0.5%, preferably 0.015-0.4%, most preferably 0.02 -0.2%, for example 0.06% of LC-PUFAs.

Since the present invention relates to a nutritional composition, it preferably comprises other constituents, such as macro- and micronutrients, functional food ingredients, for example. Preferably, constituents other that unsaturated fatty acids and encapsulated trace elements are part of component (a) of the present composition.

For example, component (a) further comprises some or all macronutrients that are present in the specific powdered composition according to the invention. For example, component (a) further comprises at least 50%, preferably at least 70%, more preferably at least 90% and most preferably at least 95% of all macronutrients of the composition according to the present invention.

Component (a) may, for example, comprise further lipids. Typical lipid source that may be used include milk fat, safflower oil, egg yolk lipid, canola oil, olive oil, coconut oil, palm oil, palm kernel oil, palm olein, soybean oil, sunflower oil, for example.

Component (a) may also comprise medium chain triglycerides (MCT), which are defined herein as triglycerides comprising fatty acids with acyl chains of 6-12 carbon atoms (C6-C12).

Generally, fatty acids are preferably present in the form of triglycerides. They may, however, also be present in the form of free fatty acids, esters of other alcohols than glycerol or in the form of phospolipids, for example.

If component (a) comprises further lipids, the lipids may provide 30-50%, preferably 35-45% of the energy of the powdered nutritional composition. In percent by weight of the powdered, dehydrated products, the composition comprises 15-30%, preferably from 20-28% of lipids.

Preferably, component (a) is essentially free of iron and copper. “Essentially free” means that the powdered, dehydrated composition comprises 0.5 mg of copper and/or iron or less.

In an embodiment of the present invention, component (a) further comprises a protein source and/or a carbohydrate source. Generally, any protein source and/or carbohydrate source suitable as ingredients in nutritional compositions may be used.

The dietary protein, which may be used may be any suitable dietary protein; for example animal proteins (such as milk proteins, meat proteins and egg proteins); vegetable proteins (such as soy protein, wheat protein, rice protein, and pea protein); mixtures of free amino acids; or combinations thereof. Milk proteins such as casein and whey protein are particularly preferred. The protein may be intact, hydrolysed protein, partially hydrolysed protein, free amino acids or a mixture of these.

The protein source preferably provides from about 7 to 25% of the energy of the composition, more preferably 7-15%, most preferably 8-13%.

If the nutritional formula includes a carbohydrate source, any carbohydrates suitable for use in nutritional compositions may be used, for example digestible carbohydrates, such as maltodextrin, maltose, sucrose, lactose, glucose, fructose, corn syrup, corn syrup solids, rice syrup solids, starch, such as cereal starch, rice starch, corn starch, and mixtures thereof.

The carbohydrate source preferably provides about 30% to about 70%, preferably 40-60% of the energy of the nutritional composition, for example if it is a complete nutritional composition.

Dietary fibre (non-digestible carbohydrates) may also be present in the nutritional composition according to the present invention if desired. Numerous types of dietary fibre are available. For example, oligosaccharides, such as fructo-oligo-saccharides, galactooligosaccharides, xylo-oligosaccharides, fuco-oligosaccharides, manno-oligosaccharides, just to mention a few, may be added.

On the other hand, soluble and non-soluble non-starch polysaccharides may be added. Examples for soluble non-starch polysaccharides inulin, pectin, β-glucans, pectin, gum arabic, tragacanth, mucilages, guar and locust bean gum, agar, carageenans, alginates, xanthan and the like. These can be commercially obtained in a purified form or as a raw material comprising high amounts of one or several fibres. For example, pea inner fibre (pea cellular walls) comprises about 40% of pectin, about 15% of cellulose and about 45% of hemicellulose and is thus an ideal raw material providing several fibre-classes.

Suitable insoluble non-starch polysaccharides that may be added to the nutritional composition according to the invention are cellulose, hemicellulosebranched arabino-xylans or galactans, for example. Cellulose and hemicellulose are present in numerous raw materials, such as soy beans, yellow pea outer fibre (pea hull fibre), cereal bran, and/or oat hull, for example. The composition according to the invention may also include other classes of dietary fibre, such as lignin and/or resistant starch.

In an embodiment of the composition according to the present invention, the component (a) further comprises at least one anti-oxidant. Preferably, this antioxidant is wet-added. Suitable antioxidants are, for example vitamin C, Tocopherol and other food-grade antioxidants. Vitamin C is particularly preferred, because it is suitable also for addition in infant formulae. The antioxidant is added in an amount that sufficiently protects unsaturated fatty acids from oxidation. Preferably, the composition comprises 40-1000 mg, more preferably 50-200 mg of vitamin C per 100 g of the powdered, dehydrated composition.

In a preferred embodiment, the composition according to the present invention comprises, in addition to components (a) and (b), an antioxidant, which is dry-added to the nutritional composition. The antioxidant of component (a) and the dry-added antioxidant may be different or the same. Preferably, vitamin C is used in both instances.

The powdered composition may comprise further ingredients, which are designed to meet the nutritional needs of the particular human being, or provide further benefits or functionalities. For example, the composition is preferably “nutritionally complete”, that is it contains adequate nutrients to sustain healthy human life for extended periods. Preferably, the composition comprises vitamins and minerals. Also trace elements that are not encapsulated, because they do not strongly interact with lipid oxidation may be supplied.

If necessary, the composition may contain emulsifiers and stabilisers such as soy lecithin, citric acid esters of mono-and di-glycerides, and the like.

The composition may optionally contain other substances, which have a beneficial effect, such as lactoferrin, nucleotides, nucleosides, and the like.

For example, the nutritional composition may further comprise a probiotic micro organism, selected from the geni Bifidobacterium, Lactobacillus, Strepotococcus, and mixtures of these.

As with the further ingredients that may be present in component (a), also the preparation of the powdered nutritional composition according to the present invention may be performed by the skilled person in a great number of suitable ways and the manufacturer is given various possibilities to adapt the process to specific requirements, imposed by certain ingredients, for example, and to modify the end-product according specific preferences and the type of nutritional composition to be produced.

For example, the powdered composition may generally be obtained by preparing a dried or powdered nutritional composition comprising unsaturated fatty acids according to the state of the art, and by adding encapsulated trace elements, component (b), to the dried composition.

According to a preferred embodiment, component (b) is dry-added to the remaining components of the nutritional composition.

For example, WO 01/1990 discloses the preparation of a powdered infant formula on pages 9-10, said composition optionally also comprising LC-PUFAs.

On the other hand, EP 1 010 374 discloses, starting on page 4 line 56 and extending to page 5, line 20, a process for preparing a powdered composition, which is rich in fibre.

Generally, powdered compositions are prepared by hydrating solid ingredients, mainly macronutrients, such as carbohydrates, a protein source, fibre, etc, if present, in a suitable vessel, to obtain a hydrated mixture. If some ingredients are difficult to dissolve in cold water, these are added first to hot water and then the hydrated mixture may be cooled somewhat to lower temperatures for addition of further ingredients, for example. The hydrated mixtures may also be referred to as the “wet-mix”.

If vitamins and minerals are provided, they may be added at this stage or also later, for example after an optional heat treatment, in case heat-sensitive vitamins are used.

If at least one antioxidant is added to be part of component (a), it is added preferably before the unsaturated fatty acids are added.

In an embodiment of the method for preparing the powdered composition according to the present invention, the hydrated mixture is obtained by
adding an antioxidant to a liquid containing at least water, and,
adding to the liquid, thereafter, unsaturated fatty acids and optionally other nutrients.

Further ingredients of the nutritional composition may thus be added before, after or at the same time as the antioxidant, the latter being added prior to adding unsaturated fatty acids. In so doing, the inventor found that the protective properties of the antioxidant are more apparent.

More preferably, the antioxidant present in component (a) of the composition is added before the optional protein and/or the carbohydrate source are added.

Lipids, and in particular unsaturated fatty acids of component (a) may be added directly to a hydration vessel or in-line into the tube transporting the hydrated mixture to a heating or a homogenisation tank, for example. Generally, lipids are added at a relatively late stage due to their insolubility in water or their need of being emulsified to provide stable hydrated mixtures.

A thermal treatment, such as a UHT, may be conducted to reduce bacterial load. For example, the hydrated mixture may be rapidly heated to a temperature in the range of about 70° C. for 5 minutes to about 110° C. for 3 seconds. This may be carried out by steam injection or by heat exchange, for example with a plate heat exchanger.

Homogenisation may be conducted once all ingredients of component (a) are present. Suitable homogenisation parameters (pressure, temperature) are known to the skilled person, may be deducted from the prior art and are dependent from the particular formula to be produced.

After homogenisation, the powder may be obtained by drying, such as spray drying, roller drying or other suitable drying processes, for example, to obtain powder-component (a) according to the present invention.

Component (b) may then be added in suitable amounts in the form of particles of iron encapsulated with or in a fat, wax or mixtures thereof to the powdered component (a), to obtain the powdered composition according to the present invention. The amount of component (b) depends on the amount of iron and its bio-availability and may be added in the ranges as already indicated above.

Preferably, the method according to the present invention for preparing a powdered nutritional composition comprises the further step of packing the powdered composition into packs under oxygen-reduced conditions.

The different powder components (a) and (b) constitute different particles of the nutritional composition or different parts of particles and their presence and amounts may be determined by microscopy.

The powdered composition may easily be reconstituted by a particular consumer, for example. This may be done by adding a watery liquid, such as water or milk, and stirring. Preferably, water is heated above 100° C. to kill potential pathogens and is then left to cool to less than about 50° C., preferably 40° C., before mixing with the powdered composition. If higher temperatures are used, the oxidation stable lipids that may be used as encapsulation matrix of the trace elements risk to rapidly melt, followed by extensive oxidation of unsaturated fatty acids.

According to the above-disclosed methods, the powdered composition according to the invention contain unsaturated fatty acids, such as LC-PUFAs, in a powdered component (a) and an iron source encapsulated with or in a fat, wax or mixtures thereof, which is thereby physically separated from the LC-PUFAs in the form of a further powder component (b), both components constituting different particles of the powdered composition. The unsaturated fatty acids may further be protected by the presence of an antioxidant in powder component (a).

In so doing, off-taste development in powdered nutritional compositions comprising unsaturated fatty acids is strongly reduced and prolonged shelf-life is achieved.

FIG. 1 schematically shows powder particles that fall under the definition of the composition according to the present invention. Component (a) is a particle (1), which is a powder-matrix comprising unsaturated fatty acids and which may further comprise an anti-oxidant (3), together with other constituents, such as macro-nutrients (lipids, proteins) within the particle forming component (a).

Encapsulated iron (2) may be present as FeSO₄ coated by an oxidation stable fat or wax. The composition may further comprise a dry-added antioxidant (4), which is present outside the powder particles (1) and thus closer to the encapsulated iron (2).

FIG. 2 shows off-taste development in different powdered compositions A-F containing LC-PUFA after 6, 17 and 30 weeks. Composition A is the only one that is free of FeSO₄ and serves as control.

Composition B of FIG. 2 is a LC-PUFA enriched wet-mixed powder to which FeSO₄ (not encapsulated) and vitamin c were dry-added. Off-taste was strongly promoted with this composition.

Composition C of FIG. 2 is a LC-PUFA enriched, wet-mixed powder (free of vitamin C) to which iron encapsulated according to the invention and vitamin C was dry-added. Off-taste is drastically reduced.

Composition D of FIG. 2 is an LC-PUFA enriched, wet-mixed powder, to which FeSO₄ was added. In addition, Vitamin C was added in the wet-mix and as a dry powder at the end (dry-mixed). This powder showed medium off-taste development.

Composition E of FIG. 2 is an LC-PUFA enriched powder with dry added encapsulated iron, to which Vitamin C was added twice, once to the hydrated mixture of component (a) (wet-mixed), and secondly at the end, dry mixed. Off-taste is strongly reduced over the whole experimental period of 30 weeks.

Composition F of FIG. 2 shows off-taste development of an LC-PUFA enriched composition further comprising FeSO₄ (not encapsulated) and vitamin C, wherein the powder is obtained by wet-mixing all ingredients followed by spray-drying. It can be seen that albeit vitamin C is present, off taste after 17 and 30 weeks is highly developed.

### EXAMPLE 1

### Preparation of a Nutritional Composition

A nutritional composition for infants was prepared by using the components listed below. Amounts are indicated in percent by weight of dry matter.

| | |
|---|---|
| Whey protein mix, Lactose and | 50.7 |
| minerals¹ | |
| Vegetable fats | 25.5 |
| Skimmed milk | 10.2 |
| LC-PUFA Oil mix² | 1.3 |
| Lecithin | 0.2 |
| Lactose | 9.4 |
| Micronutrients | 0.6 |
| Sodium ascorbate | 0.1 |
| Water | 2.0 |

¹The minerals comprise sodium, potassium, magnesium, calcium, manganese, selenium, iodine and are free of iron and copper.
²The LC-PUFA Oil mix is a commercially obtainable oil mix, which has about 20% by weight of LC-PUFAs.

The composition is prepared by dissolving sodium ascorbate (antioxidant) in water and adding all other components thereto, with the exception of the LC-PUFA oil mix to prepare a hydrated mixture. Thereafter, the LC-PUFA oil mix and the vegetable fats are added in line, before a heat treatment of 100° C. for 15 seconds.

The mixture is homogenised at 100 bar and 80 ° C. and then spray dried. The composition is further supplied with a powdered preparation of probiotic bacteria, which made up only a very small contribution to the total dry weight of the composition.

The spray dried powder is then supplied with encapsulated iron obtained from Dr. Paul Lohmann GmbH KG, Emmerthal, Germany. Per 100 g of powder obtained after spray-drying, 0.1 g of encapsulated material (including FeSO₄ and oxidation stable fats) was added.

Thus, a powdered composition comprising, in percent by weight, 0.05% of FeSO₄ and 0.13% of LC-PUFAs (ARA and DHA) is obtained.

The powdered infant composition was stored for 30 days without noticeable development of typical “fishy” off-flavour.

### EXAMPLE 2

### Sensory Tests With LC-PUFA and Iron-Containing Nutritional Compositions

For a comparative sensory test, six different powdered nutritional compositions (A-F) comprising all the same amount of LC-PUFAs but varying sources of iron and vitamin C were prepared. The compositions were also varied in the way the iron or antioxidant was added (dry-mixed or wet-mixed).

Standard composition for sensory trial (percentages by weight):

| | |
|---|---|
| Lactose | 40.3 |
| Vegetable fat mix | 25.6 |
| Demineralized whey | 15.6 |
| Milk powder | 9.0 |
| K-Caseinate | 4.0 |
| Fish oil (20% DHA)¹ | 0.22 |
| Lecithin | 0.6 |
| Sodium Ascorbate | 0.1 |
| Micronutrients (without iron) | 1.7 |
| Water | 3.0 |

¹The fish oil was commercially obtained from Sofinol under the tradename Fishoil type NIF.

The compositions were prepared according to the same procedure as given in Example 1.

The six powdered compositions were supplemented with iron/antioxidant according to the protocol below (see also comments of FIG. 2):

A: without any iron (reference sample),

B: non-encapsulated (ne-)iron and vitamin C dry mixed,

C: encapsulated iron and vitamin C dry-mixed,

D: vitamin C wet-and dry mixed and ne-iron dry-mixed only,

E: vitamin C wet-and dry mixed and encapsulated iron dry-mixed

F: all wet-mixed, including encapsulated iron and vitamin C.

If iron was added (Formulas B-F), the composition contained 0.025 g of FeSO₄ per 100 g of dehydrated powder.

The comparative sensory profile (simplified profile) was carried out with a trained external panel of 16 persons. Each taster was given a set of samples in pairs (one reference A and one coded samples, selected from B-F). The tasters were then asked to compare the reference with the coded sample for sensory attributes. These attributes include off-odours and off-flavours such as foreign odour, oxidised flavour and have been previously defined by the panel.

A 9-point scale was used (from −4 to +4) where the reference is positioned at 0. The samples were tasted blind and in randomised order.

The test compositions were stored for different intervals (6, 17, 30 weeks), in order to assess the influence of time on off-flavour development. The sensory tests have been performed using the same methodology as described.

The results of the sensory test are given in FIG. 2, which indicates huge differences in the development of off-flavour depending on the form of iron supplementation and processing of compositions A-F.

The lowest mean off taste was obtained with the powdered composition D, which contained dry-added iron in an encapsulated form and an antioxidant (vitamin C), which was wet-mixed.

1. A powdered nutritional composition, which contains a powder component (a) comprising unsaturated fatty acids and an antioxidant, and, a further powder component (b) comprising at least one trace element encapsulated with or in a fat, wax or mixtures thereof.

2. The composition according g claim 1, wherein the trace element is iron, cobalt, cupper or mixtures thereof, in a bio-available form.

3. The composition according to eceding claim 1 or 2, wherein component (a) further comprises a protein source and/or a carbohydrate source.

4. The composition according to any of the preceding claims, which comprises, in addition to components (a) and (b), an antioxidant, which is dry-added to the nutritional composition.

5. The composition according to any of the preceding claims, wherein component (b) is dry-added to the remaining components of the nutritional composition.

6. A method for preparing a powdered nutritional composition comprising unsaturated fatty acids and trace-elements, comprising the steps of:
preparing a hydrated mixture comprising unsaturated fatty acids and an antioxidant to obtain a component (a),
drying the hydrated mixture to obtain a powder component (a),
adding a powder component (b), which comprises at least one trace element encapsulated with or in a fat, wax or mixtures thereof.

7. The method according to claim 6, wherein the hydrated mixture is obtained by
adding an antioxidant to a liquid containing at least water, and,
adding to the liquid, thereafter, unsaturated fatty acids and optionally other nutrients.

8. A method of reducing off-flavour development in a powdered nutritional composition which contains unsaturated fatty acids and an iron source by protecting the unsaturated fatty acids in a powder component (a) comprising an antioxidant and maintaining the iron source encapsulated with or in a fat, wax or mixtures thereof outside the individual particles of the powder component.
